(19) United States
(12) Patent Application Publication
(10) Pub. No.: US 2008/0205589 A1
(43) Pub. Date: Aug. 28, 2008

(54) Clinical workflow for visualization and management of catheter intervention

(76) Inventor: Estelle Camus, Mountain View, CA (US)

Correspondence Address: BRINKS HOFER GILSON & LIONE, P.O. BOX 10395, CHICAGO, IL, 60610, US

(21) Appl. No.: 12/072,267

(22) Filed: Feb. 25, 2008

Related U.S. Application Data
(60) Provisional application No. 60/904,134, filed on Feb. 27, 2007.

(51) Int. Cl. A61B 6/03; G06Q 50/00

(52) U.S. Cl. 378/20; 705/2

(57) ABSTRACT

A method of diagnosis or treatment of a patient in a catheterization laboratory is described, the method including the soliciting of advice from an expert located remotely from the catheterization laboratory. The expert is contacted and requested to log on to a client computer in communications with a computer in the catheterization laboratory, so that image and other data related to the patient may be displayed at the remote location. The access rights to the data may be established and limited by the catheterization laboratory or a hospital data management system. The expert may view and may manipulate the data provided, control a catheter, or may request additional data or adjust the angiographic imaging device, and may offer an opinion on the diagnosis or treatment as requested by personnel of the catheterization laboratory.

This application claims the benefit of priority to U.S. provisional application 60/904,134, filed on Feb. 27, 2008, which is incorporated herein by reference.

### TECHNICAL FIELD

The present application relates to clinical workflow in a catheterization laboratory.

### BACKGROUND

Therapeutic interventions in the catheterization laboratory comprise two main steps: diagnosis, and treatment.

Typically, at least two physicians (for instance, a resident and an intern) may work together in the catheterization laboratory and exchange their opinions: for example, in diagnosis and choosing a therapy. However, it is not uncommon that the opinion of an expert (such as the chief physician) is necessary to arrive at the final diagnosis, or to obtain advice regarding a difficult treatment so as to be able to perform the treatment.

The expert whose consultation is desired by the catheterization laboratory staff is usually called by phone or paged. This action may result in a substantial period of time elapsing before the expert arrives at the angiography lab to review the (image) data for the patient and provide an opinion or advice. This type of delay is exacerbated if the expert is on a ward in the hospital that is far away from the catheterization laboratory, or in a similarly inconvenient location. Usually, a telephone call is not sufficient, since the expert cannot see the image data, and such data are difficult to describe on the telephone.

In a cardiac catheterization laboratory rapid decisions are may be necessary to improve the probability of a satisfactory outcome. Quick decisions are the key to a successful outcome for the patient: such decisions may include ascertaining the location and severity of a stenosis and using an appropriate stent for opening of a narrowed blood vessel.

Interventional radiology may be used so as to avoid more invasive surgical treatment, such as for a tumor. Again, the advice or opinion of another experienced physician is desirable. In electrophysiology, where the time duration of the procedures is long in the more complex cases, additional delays are to be avoided so that the overall length of the procedure is kept within tenable time limits. Decisions as to the appropriate therapy, such as ablation therapy, must be made as quickly as possible within the diagnostic protocol.

For other modalities, such as for magnetic resonance tomography, solutions to this problem exist, such as the “Expert-i” product from Siemens Medical Solutions. With this solution, the expert can log in on a client computer having a data connection with the MRI system and display the images and parameters on the computer screen in real time. The expert can talk on the phone with the personnel in the MRI suite and give them advice for continuing the examination, adjusting the parameters, and so forth.

### SUMMARY

A platform for remote visualization and remote control of an angiography system and the corresponding clinical workflow is described.

In an aspect, a method of performing a catheterization procedure includes, providing a catheterization laboratory, the laboratory including at least an X-ray device and a catheter, and obtaining images of a patient. The images may be displayed in the catheterization laboratory or in proximity thereof. When a further opinion is desired from a person not located in the catheterization laboratory, the catheterization laboratory requests the person to log on to a client computer having an interface with a communications network, providing the remotely located person with access to image and other patient data.

In another aspect, a method of diagnosing a patient from a remote location, includes a person at a remote location receiving a request from a catheterization laboratory. The person may log on to a client computer in communications with a computer in the catheterization laboratory so as to view image and other data associated with the patient, and may request additional patient images or other data. The communications between the remotely located person and personnel of the catheterization laboratory may include text or voice. As a result of viewing the data, the remotely located person may provide a suggested diagnosis or treatment for the patient.

In yet another aspect, a method of treating a patient in a catheterization laboratory includes receiving a patient in the catheterization laboratory, and preparing the patient for an angiogram and obtaining angiographic images. A determination may be made as to whether another “expert” opinion is desired or required, and a person suitable for performing the function is contacted. When the person is not in the catheterization laboratory, or cannot come to the catheterization laboratory in a timely manner, the expert may be requested to log on to a client computer in communication with a computer in the catheterization laboratory, so that the expert may view or manipulate the angiographic data. The expert may provide a diagnosis or recommended treatment plan orally or by means of a text message, or a graphical user interface (GUI).

In a further aspect, a computer-readable medium has instructions executable on a computer stored thereon, the instructions causing a computer system to perform a method of treating a patient, including obtaining angiographic image data; and obtaining an expert opinion by: communicating with an expert located outside of the catheterization laboratory; requesting the expert to log on to a client computer in communication with a computer in the catheterization laboratory; enabling the expert to at least one of view or manipulate the angiographic image data; and receiving an opinion from the expert.

### BRIEF DESCRIPTION OF THE DRAWINGS

FIG. 1 is a block diagram of the platform for performing the workflow of a catheterization procedure; and

FIG. 2 is a flow chart of the main steps in obtaining an opinion from a person not located in the catheterization laboratory.

### DETAILED DESCRIPTION

Exemplary embodiments may be better understood with reference to the drawings. Like numbered elements in the same or different drawings perform equivalent functions.

The combination of hardware and software to accomplish the tasks described herein may be termed a platform. The instructions for implementing processes of the platform may be provided on computer-readable storage media or memories, such as a cache, buffer, RAM, removable media, hard drive or other computer readable storage media. Computer readable storage media include various types of volatile and nonvolatile storage media. The functions, acts or tasks illustrated or described herein may be executed in response to one or more sets of instructions stored in or on computer readable storage media. The functions, acts or tasks may be independent of the particular type of instruction set, storage media, processor or processing strategy and may be performed by software, hardware, integrated circuits, firmware, micro code and the like, operating alone or in combination. Some aspects of the functions, acts, or tasks may be performed by dedicated hardware, or manually by an operator.

The platform may be a catheterization laboratory, and may include ancillary computing and telecommunications devices and networks, or access thereto. Other aspects of the platform may include a remotely located client computer. The client computer may have other functions not related to the platform described herein, and may therefore be shared between users having unrelated functions.

The computer instructions for any processing device may be stored on a removable media device for reading by local or remote systems or processors. In other embodiments, the instructions may be stored in a remote location for transfer through a computer data network, a local area network (LAN) or wide area network (WAN) such as the Internet, by wireless techniques, or over telephone lines. In yet other embodiments, the instructions are stored within a given computer, system, or device.

Where the term “data network”, “web” or “Internet” is used, the intent is to describe an internetworking environment, including both local and wide area networks, where defined transmission protocols are used to facilitate communications between diverse, possibly geographically dispersed, entities. An example of such an environment is the world-wide-web (WWW) and the use of the TCP/IP data packet protocol, and the use of Ethernet or other known or later developed hardware and software protocols for some of the data paths.

Communications between the devices, systems and applications may be by the use of either wired or wireless connections. Wireless communication may include, audio, radio, lightwave or other technique not requiring a physical connection between a transmitting device and a compatible receiving device. While the communication may be described as being from a transmitter to a receiver, this does not exclude the reverse path, and a wireless communications device may include both transmitting and receiving functions. A wireless communications connection may include a transceiver implementing a communications protocol such as IEEE 802.11b/g, or the like such that the transceivers are interoperable.

The platform makes it possible for the expert, who may be at a remote location, such as a ward, an office or off site, to connect (log on) to the imaging system and the workstation of the catheterization lab from a computer terminal (client computer) over a data network, which may be a local area network (LAN), a wide area network (WAN) or the Internet. With this connection, image data and other information can be exchanged between the expert at the client computer and the staff in the catheterization or angiography lab.

Where the term “client” is used, a computer executing a program of stored instructions and accepting input from a person, and displaying data, images or the like, in response to such input is meant. Corresponding to the client is another computer, the “server”, that retrieves the data, images, or the like in response to requests received from the client, and transmits the data as information over a communications network. It will be understood by persons of skill in the art that often a computer may act as both a client and a server, and that networks may have intermediate computers, storage devices and the like to provide the functional equivalent of a client and a server interaction protocol. There is no implication herein that any of the functions capable of being performed by a digital computing device, including storage and display devices is restricted to being performed on a specific computer, or in a specific location, even though the description may use such locations or designations for clarity in the examples provided.

A clinical workflow for angiography or catheterization includes: patient preparation and input of the patient identification data, as applicable; beginning the intervention by obtaining access to veins or arteries; obtaining initial angiographic images; and, determining whether the advice of an expert not currently located in the treatment room is needed to provide advice, consultation or a decision on the diagnosis or the appropriate course of treatment.

The term “expert” is intended to encompass any person not physically located in the catheterization laboratory where the procedure is being performed. This may include physicians, equipment consultants, or the like.

The clinical workflow may include one or more of the steps of; laboratory personnel contacting the expert directly by cell phone, or using a pager or other personal communications device; the expert responding by logging on to a client computer at a remote location; accessing the data suite of the catheterization laboratory as enabled in accordance with the access rights protocol of the hospital, and such data as may be remotely displayed may be either selected by the catheterization laboratory staff or by the expert for display on the remotely located client computer. The same data may be displayed on a monitor local to the catheterization laboratory. The data may include real-time angiographic images from the imaging system, and data from the angiographic workstation including vital signs, electrocardiogram (EKG) traces, and the like.

Voice and, optionally, data communication may be established between the catheterization laboratory and the remotely located expert, and the expert and the catheterization laboratory may coordinate the display and interpretation of diagnostic images and other data. With the appropriate access rights, the expert may be permitted to modify data or images on the workstation or on the imaging system

After reviewing the data, the expert may give the clinical personnel in the catheterization laboratory an assessment; this may be performed over a telephone connection or by text input on the client computer, or by a voice-over-data protocol. At the conclusion of the consultation the expert may log off from the connection or be logged off by the personnel in the catheterization laboratory.

As the expert may be in a location other than the catheterization laboratory, the identification of the expert as a person having access rights to the data and images in the present case must be established. This would be considered an appropriate practice in any digital communications system, but is particularly relevant in a hospital setting where laws and regulations have been promulgated to protect the privacy of patients, and information relating to patients. The log-on process may include, for example, the use of a log-on name and password, and may also make use of biometric data, an ID badge, a video imaging device, or the like so as to ensure that the appropriate individual is being granted access.

Once logged on to the client computer, the expert may have access to data that are generated in the catheterization laboratory, or are being used by the catheterization laboratory for the diagnosis and treatment of the patient. This data may include: stored and real-time angiographic images; 3D reconstructed imaging data and perfusion evaluation; imaging data from other imaging modalities such as intravascular ultrasound (IVUS), optical coherence tomography, carto-mapping and hemodynamic and electrophysiological parameters from sensors such as an electrocardiogram (EKG), blood oxygen sensor (SpO2), cardiac output sensor, and the like. Some of these techniques are invasive, some are non-invasive, and some are minimally invasive, and the selection of visualization modalities is a matter of professional judgment and may involve consultation with an expert not located in close proximity to the patient, who may need to review the available diagnostic data and confer with the personnel conducting the examination and treatment.

Interventional Cardiac 3-D (IC3D), is a three-dimensional diagnostic imaging tool (available from Siemens AG, Munich, Germany), enabling clinicians to quickly quantify and visualize lesions. IC3D generates a model of a vessel that can be rotated freely in space and viewed from various angles, enabling clinicians to diagnose with precision and a lack of image foreshortening effects; collect accurate length measurements of a vessel; and assess a diameter profile and related parameters, such as degree of stenosis, so that an appropriate stent length and diameter may be selected.

IC3D may use a radiographic imaging modality such as AXIOM ARTIS (available from Siemens AG, Munich, Germany) that uses a C-arm X-ray apparatus that is rotatable such that a sequence of projection X-ray images from differing geometrical aspects is obtained by an X-ray detector positioned on an opposite side of the patient from the X-ray source. The images are reconstructed by any technique such as DynaCT (available from Siemens AG, Munich, Germany) processing for realizing computed-tomographic-like (CT-like) images.

The systems and techniques described above are typical of those which are in current use; however, any diagnostic or treatment system that produces data which can be quantified, including imaging modalities, bodily function sensors, and robots which are capable of being manipulated through an electrical control interface may equally be used.

The expert may modify the data of the imaging system or the parameters of the workstation, if the user in the catheterization laboratory or the expert has been allocated the applicable access rights. These modifications are then automatically displayed in the catheterization laboratory and recorded in the system. The user in the catheterization laboratory can delete these statements or changes step-by-step, if necessary. The data exchange may be made via a high-quality connection with a high transmission rate such as a LAN, WAN or the Internet.

Communication between the expert at the client computer and the clinical personnel in the catheterization laboratory is by at least one of a telephone over conventional telephone lines, by wireless connection such as cellular telephone, WiFi (in accordance with IEEE 802.11(b), (g) or the like), or any of the known or subsequently developed means of personal communications, which may also include video images. An intranet or Internet connection using Voice over Internet Protocol (VoIP), and a headset and speaker or earphones at the client computer may also be used. The variety of communications methods is not limited to those described herein, as the function of communicating between a remotely-located expert and the clinical personnel may be implemented in any way that results in inter-personal communications.

The content of communication between the expert and the personnel in the catheterization laboratory may be stored in non-volatile memory in the report of the procedure, along with the patient images, in the catheterization laboratory. This can be done in the form of an audio file, video file or text file (depending on the source of the information). The date, time, and list of participants (the name of the expert and of the user in the catheterization laboratory) are stored in memory as well. The image or other data actually being reviewed may be stored in a synchronized manner with the voice data. This data may be associated with the image data, and the results of the examination and diagnosis stored in another data base, which may be remotely located.

The expert may view the data and images and make an assessment of the information, including selecting various image views for evaluation, requesting additional images, which may be stored or real-time images, or specific image reconstructions, which may include segmentation, superimposition of images from various imaging modalities, or the like. This aspect may include image quality evaluation, or suggesting a better orientation and parameters for making an image, or an image reconstruction.

Examples of an expert consultation or opinion would be in support of a stenosis evaluation using a modality such as IC3D, or in assessing a stenosis in the cardiac catheterization laboratory; support for using a 3D reconstruction process (such as DynaCT) or in assessing a 3D data set in interventional radiology or neurology, for instance for assessing an unexpected bleed.

More than one expert may log in contemporaneously and communicate with the user in the catheterization laboratory using a plurality of client computers at different locations. The experts may communicate with one another in a conference call, and view the same data simultaneously. One or more of the client computers may be a mobile computer, or a computer located outside of the hospital. Access to the images and data may be protected by the log-on procedure. The expert may be located in a different hospital.

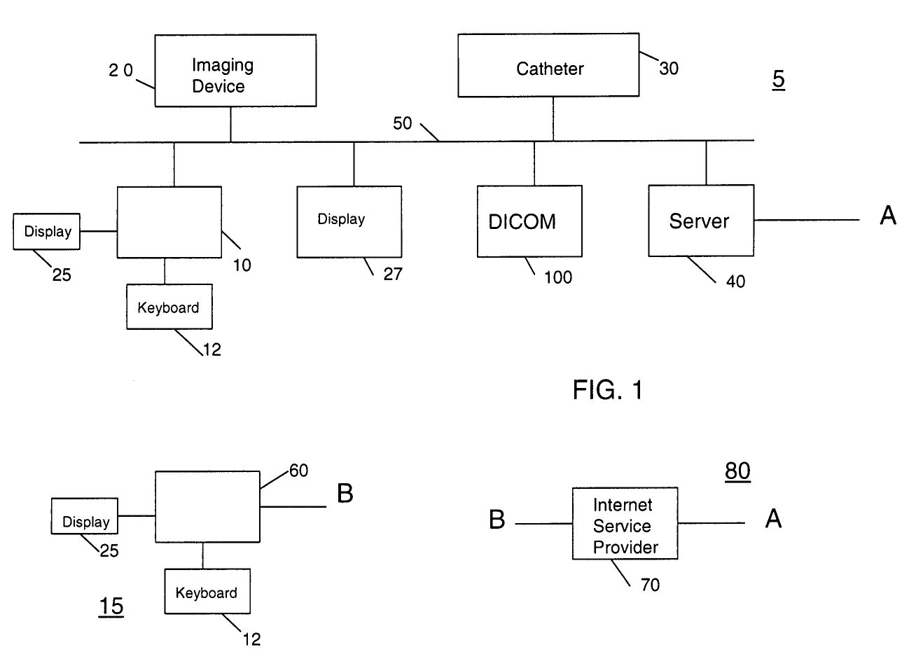

The platform used to facilitate the clinical workflow procedure disclosed herein may include, as shown in FIG. 1, a laboratory workstation 10, which is connected to a variety of laboratory diagnostic and treatment equipment 20. Although not shown in detail, the equipment 20 in a catheterization laboratory may include a C-arm X-ray device which may be used with and without the administration of contrast agents, a catheter manipulator and position sensor; vital signs monitors, an electrocardiograph (EKG) and the like. The workstation 10 may have a keyboard and display monitor 25 for control, and a plurality of display monitors 27 for displaying image data and other patient data. The laboratory workstation may connect to a DICOM (Digital communications in Medicine) workstation for the management and storage and retrieval of the image and other patient data, and may connect to a server 40 using a local area network 50. Client computers 60 located outside of the catheterization laboratory may also connect to the server 40 using the local area network (LAN) a wide area network (WAN) or the Internet. Where the client computer is located outside a “firewall” for network security, a virtual private network (VPN) may be established. The server may connect to an internet service provider using a router 70, and communicate over an external telecommunications network. The internet service provider may be a separate entity from the users, and provide the connectivity on a leased, pay-for-use, or other basis. The data may be transmitted or received over the interfaces A and B as shown in FIG. 1. The external communications environment may result in the data being modulated on a carrier wave for transmission on a network.

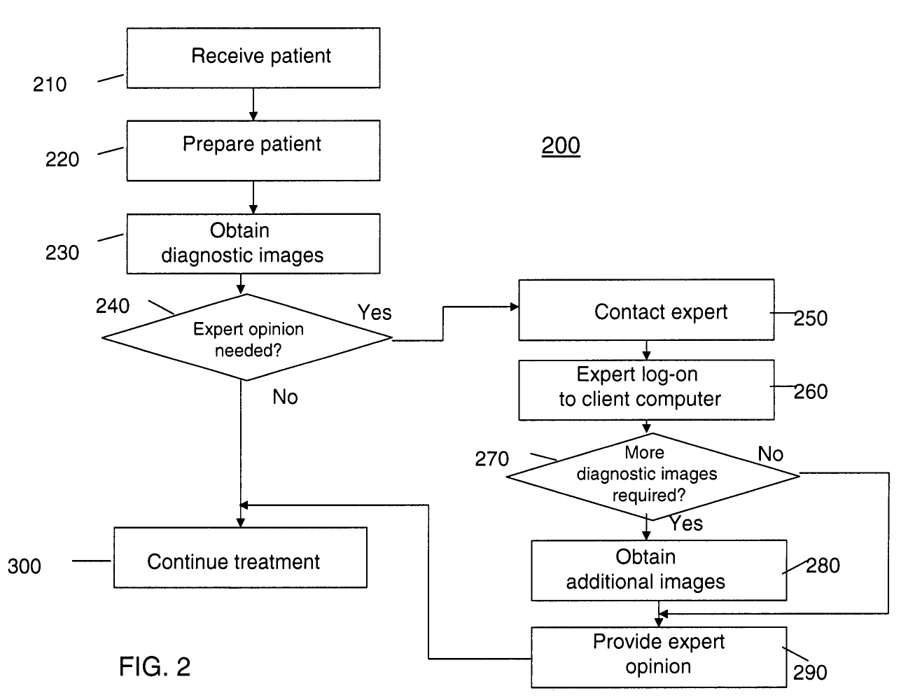

An example of the clinical workflow in a catheterization laboratory may include the steps, shown in FIG. 2, of: preparing the patient for the procedure, including entering or retrieving patient data, and preparing a sterile area on the patient; commencing the procedure by gaining access to veins or arteries and using an angiographic imaging modality to obtain initial images; and, determining whether consultation with an expert not located in the catheterization laboratory is needed in order to proceed further. If a consultation with an expert is needed, the expert is contacted; the expert may log in to a data network using a client computer; the expert may be enabled to access patient image data or other patient data based on the level of access rights accorded to the expert by the log-in or by the laboratory personnel; the laboratory personnel or the expert may cause the data to be displayed on a monitor of the client computer so that the expert may assess the data by viewing the data and by talking to the laboratory personnel if needed; the expert makes a determination of the opinion or recommendation to be provided to the laboratory personnel and provides the opinion by a text input to the client computer, or by voice communications; the expert may remain in communication with the laboratory or log off the data network, depending on the opinion given, and a determination as to the likelihood that follow-up opinions, recommendations or instructions will be needed during the procedure. After receiving the opinion of the expert, the laboratory personnel may continue with the procedure in accordance with the course of treatment or further diagnosis that has been advised. In some situations, the expert may be provided with a capability to adjust or manipulate one or more of the devices used for diagnosis or treatment by the transmission of control data from the remotely located computer.

In a further aspect, the computer console may provide access to the catheterization laboratory for personnel being trained as physicians or technicians, so that the diagnostic steps may be observed and the personnel being trained may be afforded voice communication with the laboratory personnel or an instructor.

While the methods disclosed herein have been described and shown with reference to particular steps performed in a particular order, it will be understood that these steps may be combined, sub-divided, or reordered to from an equivalent method without departing from the teachings of the present invention. Accordingly, unless explicitly stated, the order and grouping of steps is not a limitation of the present invention.

Although only a few examples of this invention have been described in detail above, those skilled in the art will readily appreciate that many modifications are possible without materially departing from the novel teachings and advantages of the invention. Accordingly, all such modifications are intended to be included within the scope of this invention as defined in the following claims.

What is claimed is:

1. A method of performing a catheterization procedure, the method comprising:
providing a catheterization laboratory, including at least an X-ray device and a catheter;
obtaining images of a patient;
communicating with a person remotely located from the catheterization laboratory to request the person to log on to a client computer having an interface with a communications network; and
enabling the person to access image and other patient data obtained by the X-ray device by transmitting the image data over the interface with the communications network.

2. The method of claim 1, further comprising retrieving image data previously obtained for the patient in response to a request message received from the client computer and transmitting the retrieved image data to the client computer over the interface with the communications network

3. The method of claim 1, further comprising recording data and voice communications between the catheterization laboratory and the remotely located person, wherein the time relationship of the data and voice communications to the image and patient data being accessed is identified.

4. The method of claim 1, wherein the catheterization laboratory is in communication with a plurality of remotely located client computers over the interface with the communications network.

5. The method of claim 4, wherein the plurality of remotely located persons are in communication with each other using the interface with the communications network located at the catheterization laboratory location.

6. The method of claim 1, wherein at least one of the X-ray device or the catheter is manipulated in response to control data received over the interface with the communications network.

7. A method of diagnosing or treating a patient from a remote location, the method comprising:
receiving a request from a catheterization laboratory;
logging on to a client computer in communications with a computer in the catheterization laboratory, the communications being performed through an interface of a communications network;
receiving and displaying image data and other data associated with a patient in the catheterization laboratory;
at least one of requesting additional patient images, receiving additional patient images, or performing interpersonal communications with personnel of the catheterization laboratory; and
providing a diagnosis based at least in part on the received patient images and other data.

8. The method of claim 7, wherein the interface with the communications network provides for voice and data communications.

9. The method of claim 7, wherein the communications network interface is a data communications interface and a voice communications interface.

10. The method of claim 7, wherein the image data is an angiographic image

11. The method of claim 10, further comprising: manipulating at least one of a real-time angiographic image or a catheter.

12. The method of claim 7, further comprising recording data and voice communications between the catheterization laboratory and the remote location, wherein the time relationship of the data and voice communications to the image and patient data is identified.

13. A method of treating a patient in a catheterization laboratory, the method comprising:
receiving a patient in the catheterization laboratory;
entering or retrieving data relating to the patient;
preparing the patient for an angiogram;
obtaining angiographic image data;
determining whether another expert opinion is needed;
obtaining the another expert opinion by:
communicating with an expert located outside of the catheterization laboratory;
requesting the expert to log on to a client computer in communication with a computer in the catheterization laboratory;
enabling the expert to at least one of view or manipulate the angiographic image data; and
receiving an opinion from the expert.

14. The method of claim 13, further comprising performing a specific treatment based on the opinion.

15. The method of claim 13, wherein the communication between the communications laboratory and the client computer is through an interface of a communications network.

16. The method of claim 14, wherein the communications network is at least one of a local area network or a wide area network.

17. The method of claim 16, wherein the wide area network is the Internet.

18. The method of claim 17, wherein the data is transmitted over the communications network by modulating digital signals on a carrier wave.

19. The method of claim 13, wherein communications between the remote location and the catheterization laboratory by at least one of data or voice is recorded so that the time relationship of the data and voice communications to the image and patient data is identifiable.

20. A computer program product, the product being stored or distributed on a machine readable medium, comprising:
instructions for causing a computer to perform a method of:
obtaining angiographic image data;
obtaining an expert opinion by:
communicating with an expert located outside of the catheterization laboratory;
requesting the expert to log on to a client computer in communication with a computer in the catheterization laboratory;
enabling the expert to at least one of view or manipulate the angiographic image data; and
receiving an opinion from the expert.
